(19) United States
(12) Patent Application Publication
(10) Pub. No.: US 2025/0321303 A1
(43) Pub. Date: Oct. 16, 2025

(54) Method and System for Determining an Orientation and an X-Coordinate of a Movable Object Relative to a B0 Field Magnet

(71) Applicant: Siemens Healthineers AG, Forchheim (DE)

(72) Inventor: Johann Sukkau, Herzogenaurach (DE)

(73) Assignee: Siemens Healthineers AG, Forchheim (DE)

(21) Appl. No.: 19/174,999

(22) Filed: Apr. 10, 2025

(30) Foreign Application Priority Data
Apr. 12, 2024 (DE) 10 2024 203 419.3

(51) Int. Cl. G01R 33/44; G01B 7/004; G01B 7/31; G01R 33/30

(52) U.S. Cl. CPC G01R 33/443; G01B 7/004; G01B 7/31; G01R 33/307

(57) ABSTRACT

A method for determining orientation of a movable object relative to a B0 field magnet in a magnetic resonance tomography device using a three-dimensional magnetic field strength sensor fixed to the object. The method involves providing B0 reference data containing characteristic magnetic field strengths for multiple xyz-coordinates. The object is first positioned outside the B0 field magnet along the z-axis, where first measurement values of field strength components are taken. A first angle value (Yaw1) is calculated using these measurements and reference data. The object is then moved to a second position within the B0 field magnet, where second measurement values are obtained. A second angle value (Yaw2) is calculated using these new measurements and reference data. Finally, the first angle value is corrected using the second angle value to provide a corrected first angle value (Yaw1′).

### CROSS REFERENCE TO RELATED APPLICATIONS

This patent application claims priority to German Patent Application No. 10 2024 203 419.3, filed Apr. 12, 2024, which is incorporated herein by reference in its entirety.

### BACKGROUND

### Field

The present disclosure relates to a method and a system for determining an orientation of a movable object relative to a B0 field magnet of a magnetic resonance tomography device in an xz-coordinate plane by means of a three-dimensional magnetic field strength sensor arranged at a fixed relative position on the object.

### Related Art

Magnetic resonance tomography devices are imaging apparatuses which, in order to map an examination object, align nuclear spins of the examination object with a strong external magnetic field and excite them into precession about this alignment by way of an alternating magnetic field. The precession or return of the spin from this excited state into a state with lower energy in turn generates an alternating magnetic field as the response, which is received via antennas.

With the aid of magnetic gradient fields, a spatial encoding is impressed upon the signals, which subsequently makes it possible to assign the received signal to a volume element. The received signal is then evaluated and a three-dimensional imaging representation of the examination object is provided.

Usually, the spatial encoding is based on an xyz-coordinate system. In this context, the z-coordinate axis is usually defined as an axis of symmetry of the B0 field magnet through a patient tunnel of the B0 field magnet in the preferred direction of the B0 field. With the usual assembly of a magnetic resonance tomography device, the z-coordinate axis is aligned horizontally and runs centrally through the opening of the windings of the B0 field magnet through a recording region of the B0 field magnet. The object being recorded is usually brought into the patient tunnel on a patient couch in parallel with the z-coordinate axis.

Together with the z-coordinate axis, an x-coordinate axis and a y-coordinate axis span a space. In an exemplary embodiment, the coordinate axes are provided orthogonally to one another and the x-coordinate axis is aligned horizontally and the y-coordinate axis is aligned vertically.

In order to receive the signal, local antennas, known as local coils, may be used, which are arranged directly on the examination object in order to attain an improved signal-to-noise ratio. The position thereof is therefore not defined in a fixed manner in relation to the rest of the magnetic resonance tomography device, in particular in relation to the B0 field magnet, and must be captured separately. In this regard, it is known, for example, to calculate the orientation of a local coil in the horizontal plane, i.e. in the xz-coordinate plane, with the trigonometric equation Yaw=atan 2 (x-field strength components, z-field strength components). The field strength components may be captured by means of a magnetic field strength sensor. In this context, the magnetic field strength sensor is arranged at a fixed relative position on the local coil. The orientation of the magnetic field strength sensor ascertained in this manner is subsequently used to calculate the x-coordinate of the magnetic field strength sensor and thus of the local coil.

However, it is therefore only possible to ascertain the actual orientation value of the local coil in the xz-coordinate plane where the magnetic field lines run in parallel with the z-coordinate axis. This is only the case within the patient tunnel, i.e. in what is referred to as the isocenter of the B0 magnet, or outside the patient tunnel only directly on the z-coordinate axis. If the local coil is not positioned in the patient tunnel and is positioned in an offset manner in relation to the z-coordinate axis, then the orientation ascertained in this manner and the x-coordinate of the local coil is subject to a systematic error in the xz-coordinate plane. In this context, the orientation error is greater, the further away the magnetic field strength sensor is arranged from the z-coordinate axis and the closer it is arranged to the B0 magnet.

In this regard, it has emerged that there exists a need for providing a method and a system, with which it is possible to provide an orientation and thus also an x-coordinate of a local coil with a high level of accuracy, even if the local coil is arranged outside the patient tunnel and in an offset manner in relation to the z-coordinate axis.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a top view of a system (magnetic resonance tomography device) according to one or more exemplary embodiments of the disclosure.

FIG. 2 shows a side view of the magnetic resonance tomography device shown in FIG. 1.

FIG. 3 is a flowchart of a method according to one or more exemplary embodiments of the disclosure for determining an orientation of a movable object relative to a B0 field magnet of a magnetic resonance tomography device.

FIG. 4A-4B show schematic representations of the magnetic resonance tomography device in FIGS. 1 and 2, with an object at a first position and a second position, according to exemplary embodiments of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, where a connection can be wireless or wired. Functional units can be implemented as hardware, software or a combination of hardware and software.

An object of the present disclosure is to provide a solution, with which it is possible to provide an orientation and thus an x-coordinate of a local coil with a high level of accuracy, even if the local coil is arranged outside the patient tunnel and in an offset manner in relation to the z-coordinate axis.

According to the disclosure, a method is disclosed for determining an orientation of a movable object relative to a B0 field magnet of a magnetic resonance tomography device in an xz-coordinate plane by means of a three-dimensional magnetic field strength sensor arranged at a fixed relative position on the object. The method may comprise:
providing B0 reference data of the B0 field magnet with characteristic magnetic field strengths for a large number of xyz-coordinates;
positioning the object at a first position outside the B0 field magnet with regard to a z-coordinate axis;
providing first measurement values of the magnetic field strength sensor at the first position, where the first measurement values at least comprise field strength components of the B0 field;
ascertaining a first angle value by means of the first measurement values of the magnetic field strength sensor at the first position and the B0 reference data;
positioning the object at a second position within the B0 field magnet with regard to the z-coordinate axis;
providing second measurement values of the magnetic field strength sensor at the second position, where the second measurement values at least comprise field strength components of the B0 field;
ascertaining a second angle value by means of the second measurement values of the magnetic field strength sensor at the second position and the B0 reference data; and
correcting the first angle value by means of the second angle value and providing a corrected first angle value.

In other words, the present disclosure proposes, in a first step, to ascertain the orientation of the object based on the field strength components of the B0 field at a first position. In this context, the first position is located outside the B0 field magnet. As explained above, the orientation of the object ascertained in this manner is subject to a systematic error if the object is not arranged on the z-coordinate axis. The x-coordinate of the object, which is derived from this orientation of the object that is subject to an error, therefore has a systematic error.

In a second step, the orientation of the object is ascertained based on the field strength components of the B0 field at a second position. In this context, the second position is located within the B0 field magnet, i.e. in the isocenter of the B0 field magnet. The orientation of the object ascertained here can subsequently be used to correct the orientation of the object outside the B0 field magnet which is subject to an error. By means of the corrected orientation, it is subsequently possible to ascertain the actual x-coordinate of the object at the first position.

In an exemplary embodiment, the angle values at the first position and second position are ascertained by applying the following equation: angle value (Yaw)=atan 2 (x-field strength component, z-field strength component). The angle values ascertained in this manner may be assigned to a 90° quadrant in the xz-coordinate plane. Such an assignment may take place by means of a modulo function.

In an exemplary embodiment, a three-dimensional acceleration sensor is arranged with a fixed relative connection on the object. The acceleration sensor may be configured to provide an orientation of the object in relation to a y-coordinate axis at the first position and the second position. The measurement values of the magnetic field strength sensor on the xz-coordinate plane may be leveled by means of the orientation of the object at the first position and the second position. In other words, the acceleration sensor makes it possible to create a rotation matrix, in order to align the measurement values of the magnetic field strength sensor, more precisely the measured field vectors, in such a manner as though the magnetic field strength sensor were located in the horizontal xz-coordinate plane.

In an exemplary embodiment, in order to provide the corrected first angle value, the first measurement values, more precisely the field vectors, which have been leveled to the xz-coordinate plane, are rotated about the y-coordinate axis by means of the second angle value in the xz-coordinate plane in such a manner that the field vector for the x-coordinate axis is parallel to the x-coordinate axis.

Measurement values of the magnetic field strength sensor at the first position, which have been corrected by means of the corrected first angle value, may be provided. A corrected x-coordinate of the object at the first position may be provided by reconciling the corrected measurement values with the B0 reference data.

In an exemplary embodiment, the B0 field magnet surrounds a patient tunnel of a magnetic resonance tomography device. The z-coordinate axis is defined by an axis of symmetry of the B0 field magnet in the preferred direction of the B0 field, where the coordinate axes may be provided orthogonally to one another and where the x-coordinate axis may be aligned horizontally and the y-coordinate axis may be aligned vertically.

In an exemplary embodiment, the magnetic field strength sensor may be configured to capture a field strength of three components of the B0 field in three directions, which span a space, and the magnetic field strength sensor ascertains the magnetic field strength as an absolute value of a B0 field vector determined by the three components of the B0 field.

The present disclosure furthermore relates to a system for determining an orientation of a movable object relative to a B0 field magnet in an xz-coordinate plane by means of a three-dimensional magnetic field strength sensor arranged at a fixed relative position on the object. The system may comprise:
a first interface, configured to receive B0 reference data of the B0 field magnet with characteristic magnetic field strengths for a large number of xyz-coordinates;
a second interface, configured to receive first measurement values of the magnetic field strength sensor at a first position, where the first measurement values at least comprise field strength components of the B0 field;
a third interface, configured to receive second measurement values of the magnetic field strength sensor at a second position, where the second measurement values at least comprise field strength components of the B0 field;
a computer unit, which is connected to the interfaces and is configured to carry out the method cited above.

In an exemplary embodiment, the system furthermore has a three-dimensional acceleration sensor that is arranged with a fixed relative connection on the object. The acceleration sensor may be configured to provide an orientation of the object in relation to a y-coordinate axis at the first position and the second position, and the measurement values of the magnetic field strength sensor on the xz-coordinate plane may be leveled by means of the orientation of the object at the first position and the second position.

In an exemplary embodiment, the system may further comprise at least one storage means (e.g., memory), in which B0 reference data of the B0 field magnet with characteristic magnetic field strengths for a large number of xyz-coordinates and/or a large number of angle values and x-coordinates of the object at the first position and corrected angle values at the first position which correspond thereto is stored.

Furthermore, the present disclosure relates to a computer program element with instructions which, when executed on data processing devices of a data processing environment, are designed to carry out the steps of the method cited above in a system cited above.

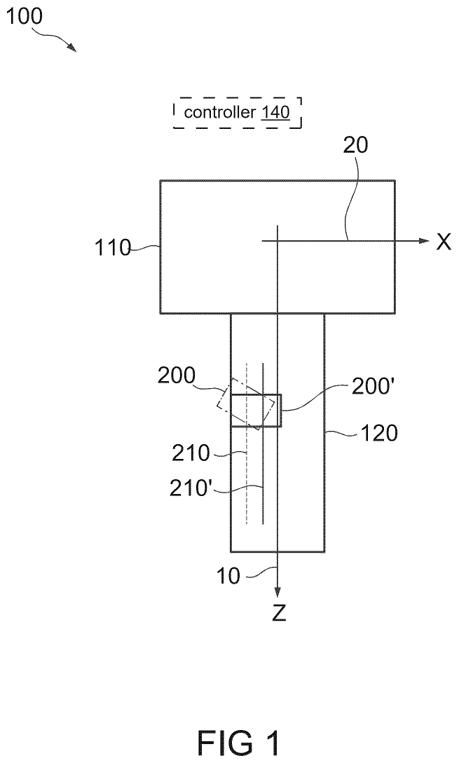

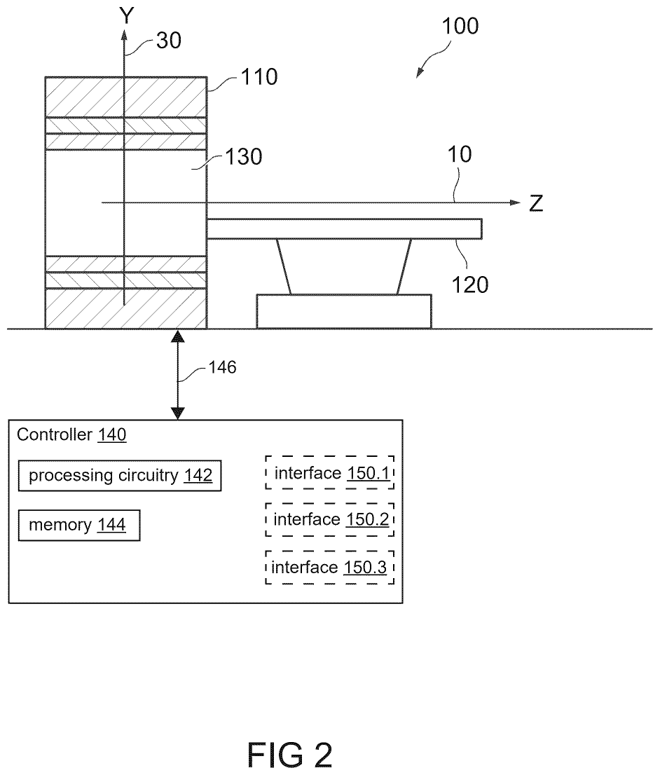

FIG. 1 shows a top view and FIG. 2 shows a side view of a system 100 according to the disclosure, in the form of a magnetic resonance tomography device 100. The magnetic resonance tomography device (scanner) 100 may comprise a B0 field magnet 110 and a patient couch 120. The scanner 100 may be controlled by a computing device, such as a controller 140. The controller 140 may be connected via one or more wireless and/or wired connections 146. Additionally, or alternatively, the controller 140 may be a component of the scanner 100, patient couch 120, and/or one or more other components of the system 100.

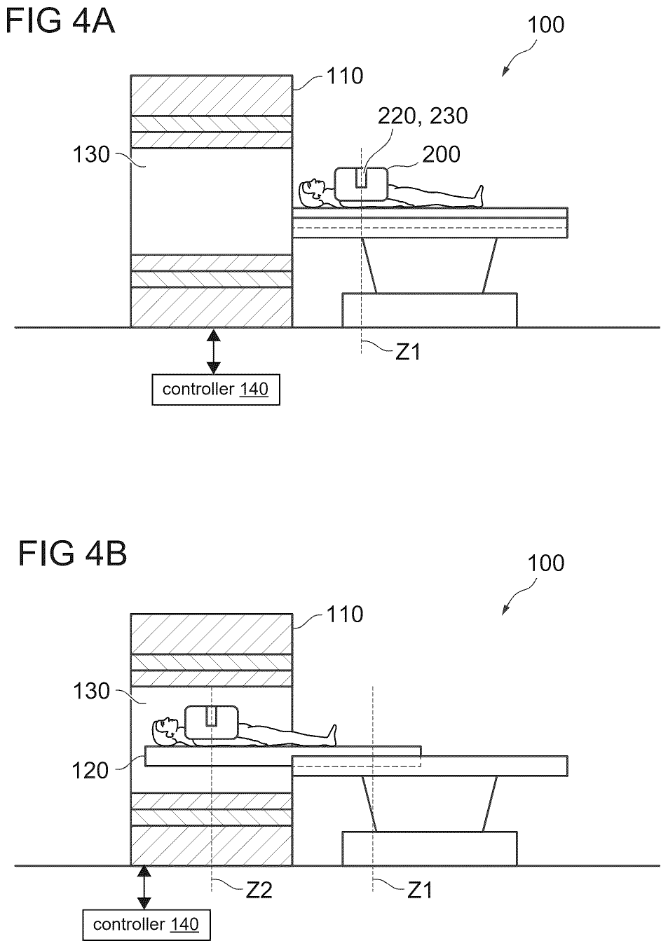

As shown in FIG. 2, the controller 140 may include processing circuitry 142 and memory 144 that may store one or more instructions and/or data. The processing circuitry 142 may execute the stored instructions (and/or receive instructions from one or more external memory units) to perform the method according to the disclosure. In an exemplary embodiment, the processing circuitry 142 may be configured to perform one or more functions and/or operations of the system 100. In an exemplary embodiment, the controller 140 may include one or more interfaces 150.1 to 150.n (e.g., three interfaces 150.1, 150.2, 150.3). The interfaces may be configured to receive B0 reference data of the B0 field magnet with characteristic magnetic field strengths for a large number of xyz-coordinates, and measurement values from one or more magnetic field strength sensors 220 (see FIG. 4A). Although the interface(s) 150 are shown as a component of the controller 140, in an exemplary embodiment, the interface(s) 150 may be additionally, or alternatively, included in one or more other components of the system 100.

As shown in FIGS. 1 and 2, the spatial encoding is based on an xyz-coordinate system. In this context, the z-coordinate axis 10, as usual, is defined as an axis of symmetry of the B0 field magnet 110 through a patient tunnel 130 of the B0 field magnet 100 in the direction of the B0 field. With the usual assembly of the magnetic resonance tomography device 100 shown, the z-coordinate axis 10 is aligned horizontally and runs centrally through the opening of the windings of the B0 field magnet 110 through the patient tunnel 130 of the B0 field magnet 110. The object being recorded is usually brought into the patient tunnel 130 on the patient couch 120 in parallel with the z-coordinate axis 10. Together with the z-coordinate axis 10, an x-coordinate axis 20 and a y-coordinate axis 30 span a space. Here, the xyz-coordinate axes may be provided orthogonally to one another, and the x-coordinate axis is aligned horizontally and the y-coordinate axis is aligned vertically.

FIG. 1 also shows an orientation of an object 200, here in the form of an exemplary local coil 200, in the xz-coordinate plane, which has been calculated by means of the trigonometric equation Yaw=atan 2 (x-field strength component, z-field strength component). In this context, the dashed representation of the local coil 200 represents the calculated orientation of the local coil 200; the representation of the local coil 200 shown by a solid line represents the actual orientation of the local coil 200. If the orientation ascertained in accordance with the above equation is subsequently used to calculate the x-coordinate of the local coil 200, then the error represented in FIG. 1 is produced in the determination of the x-coordinate. In this context, the dashed representation of the x-coordinate (see reference character 210) of the local coil 200 is based on the orientation of the local coil 200 ascertained with error; the x-coordinate shown with a solid line (see reference character 210′) represents the actual x-coordinate of the local coil 200.

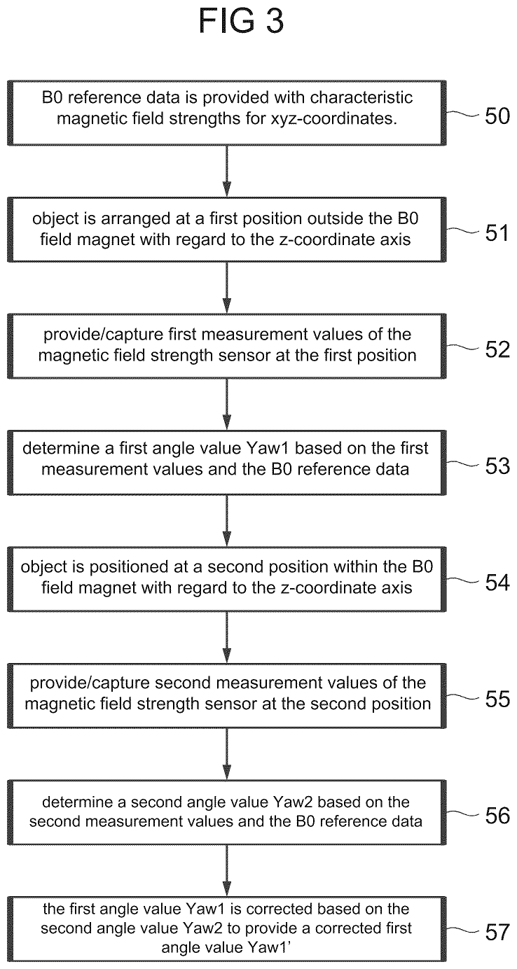

FIG. 3 shows a schematic representation of a method according to the disclosure for determining an orientation of a movable object relative to a B0 field magnet of a magnetic resonance tomography device in an xz-coordinate plane by means of a three-dimensional magnetic field strength sensor arranged at a fixed relative position on the object.

In a first step 50, B0 reference data of the B0 field magnet 110 is provided with characteristic magnetic field strengths for a large number of xyz-coordinates. This can be used when determining the position or location of the magnetic field sensor 220.

In a further step 51, the object 200 is arranged at a first position outside the B0 field magnet 110 with regard to the z-coordinate axis 10. A first position of this kind is shown in FIG. 4A, for example.

In a further step 52, first measurement values of the magnetic field strength sensor 220 are provided or captured at the first position, where the first measurement values at least comprise field strength components of the B0 field. In a further step 53, a first angle value Yaw1 is ascertained by means of the first measurement values of the magnetic field strength sensor 220 at the first position and the B0 reference data.

In a further step 54, the object 200 is positioned at a second position within the B0 field magnet 110 with regard to the z-coordinate axis. A second position of this kind is shown in FIG. 4B, for example. In a further step 55, second measurement values of the magnetic field strength sensor 220 are captured or provided at the second position, where the second measurement values in turn at least comprise field strength components of the B0 field at the second position. In a further step 56, a second angle value Yaw2 is determined by means of the second measurement values of the magnetic field strength sensor 220 at the second position and the B0 reference data. Finally, the first angle value Yaw1 is corrected in a further step 57 by means of the second angle value Yaw2 and a corrected first angle value Yaw1′ is provided. By means of this corrected angle value Yaw1′, it is possible for an actual x-coordinate of the object 200 at the first position to be provided by reconciling with the B0 reference data.

An exemplary embodiment of a method according to the disclosure is described below:

At a first position Z1 of the object 200 (see FIG. 4A), and thus at a first position of the magnetic field sensor 220 and the acceleration sensor 230, the position of the object 200 is ascertained from the measurement values of the magnetic field sensor 220 and the acceleration sensor 230. In this context, the measurement values of the acceleration sensor are first used, in order to level the B0 data measured by the magnetic field sensor 220, i.e. a rotation matrix is created, which rotates the field vectors of the magnetic field sensor 220 as though the magnetic field sensor 220 were to lie in the horizontal xz-coordinate plane.

Now, the angle value Yaw1 is calculated for the first position Z1 as follows: Yaw1=atan 2 (Hall.X, Hall.Z) and is applied for an alignment in the horizontal xz-coordinate plane.

As stated, however, it should be noted in this context that, due to the curvature of the field lines at the first position Z1, the angle value Yaw1 does not correspond to the actual orientation of the magnetic field sensor 220 (see FIG. 1). Thus, a rotation by this angle also does not lead to the x-coordinate axis of the magnetic field sensor being aligned in parallel with the x-coordinate axis.

It is now possible to normalize the angle Yaw1 by assigning the angle value to a 90° quadrant in the xz-coordinate plane; the assignment may take place by means of a modulo function. The angle value Yaw1 can therefore be assigned to one of the angle ranges. This information can already be used to ascertain a rough orientation of the object 200 and, where applicable, to already provide the B0 data for the corresponding quadrants.

The object 200 and thus the magnetic field sensor 220 and the acceleration sensor 230 are now brought into the second position Z2 (see FIG. 4B), for example by moving the patient couch 120 into the patient tunnel 130. At the second position Z2, steps above and an angle value Yaw2 for the second position Z2 are ascertained.

In this context, the second angle value Yaw2 is considerably more accurate than the first angle value Yaw1, as the field lines in the patient tunnel, i.e. in the isocenter, run in parallel with the z-coordinate axis 10. The read-in B0 reference data therefore corresponds precisely to the actual orientation of the object 200.

It is now possible to determine the x-coordinate of the object 200 at the first position more precisely, by way of the second angle value Yaw2. For this purpose, the first measurement values, which have been leveled to the xz-coordinate plane, are rotated about the y-coordinate axis 30 by means of the second angle value Yaw2 in the xz-coordinate plane so that the field strength component of the measurement values are arranged in a manner corresponding to the x-coordinate axis, in parallel with the x-coordinate axis 20. By reconciling these corrected measurement values with the B0 reference data, it is now possible for the actual x-coordinate of the object 200 at the first position Z1 to be provided.

The present disclosure is not restricted to the embodiment described above, as long as it is included by the subject matter of the following claims.

In addition, it is noted that the terms “comprising” and “having” do not exclude any other elements or steps and the indefinite article “a” or “an” does not exclude a plurality. It is further noted that features or steps, which have been described with reference to above embodiments, can also be used in combination with other features.

Moreover, it is noted that, independent of the grammatical term usage, individuals with male, female or other gender identities are included within the term.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for determining an orientation of a movable object relative to a B0 field magnet of a magnetic resonance tomography device in an xz-coordinate plane by a three-dimensional magnetic field strength sensor arranged at a fixed relative position on the object, the method comprising:
providing B0 reference data of the B0 field magnet with characteristic magnetic field strengths for a number of xyz-coordinates;
positioning the object at a first position outside the B0 field magnet with regard to a z-coordinate axis;
obtaining first measurement values of the magnetic field strength sensor at the first position, the first measurement values including at least field strength components of the B0 field;
determining a first angle value based on the first measurement values of the magnetic field strength sensor at the first position and the B0 reference data;
positioning the object at a second position within the B0 field magnet with regard to the z-coordinate axis;
obtaining second measurement values of the magnetic field strength sensor at the second position, the second measurement values including at least field strength components of the B0 field;
determining a second angle value based on the second measurement values of the magnetic field strength sensor at the second position and the B0 reference data;
correcting the first angle value based on the second angle value to determine a corrected first angle value; and
providing the corrected first angle value in electronic form as a data file.

2. The method as claimed in claim 1, wherein the first and/or the second angle values are determined as:
angle value=atan 2(x-field strength component,z-field strength component).

3. The method as claimed in claim 2, wherein the angle value is assigned to a 90° quadrant in the xz-coordinate plane.

4. The method as claimed in claim 3, wherein the assignment is based on a modulo function.

5. The method as claimed in claim 1, wherein a three-dimensional acceleration sensor is arranged with a fixed relative connection on the object, wherein the method further comprises:
providing, using the acceleration sensor, an orientation of the object in relation to a y-coordinate axis at the first position and the second position, and
leveling the first and the second measurement values of the magnetic field strength sensor on the xz-coordinate plane based on the orientation of the object at the first position and the second position.

6. The method as claimed in claim 1, wherein determining the corrected first angle value comprises: leveling the first measurement values to the xz-coordinate plane and rotating the first measurement values about a y-coordinate axis based on the second angle value in the xz-coordinate plane.

7. The method as claimed in claim 1, wherein determining the corrected first angle value comprises leveling the first measurement values to the xz-coordinate plane and rotating the first measurement values about a y-coordinate axis based on the second angle value in the xz-coordinate plane such that the field strength component of the measurement values relating to the x-coordinate axis are arranged in parallel with a x-coordinate axis.

8. The method as claimed in claim 1, wherein the first measurement values of the magnetic field strength sensor at the first position are corrected by the corrected first angle value, and an x-coordinate of the object at the first position is provided by reconciling the corrected first measurement values with the B0 reference data.

9. The method as claimed in claim 1, wherein:
the B0 field magnet surrounds a patient tunnel of the magnetic resonance tomography device,
the z-coordinate axis is defined by an axis of symmetry of the B0 field magnet in a preferred direction of the B0 field, and
the coordinate axes are provided orthogonally to one another, a x-coordinate axis being aligned horizontally and a y-coordinate axis being aligned vertically.

10. The method as claimed in claim 1, wherein the magnetic field strength sensor is configured to capture a field strength of three components of the B0 field in three directions, which span a space, and the magnetic field strength sensor ascertains the magnetic field strength as an absolute value of a B0 field vector determined by the three components of the B0 field.

11. The method as claimed in claim 1, wherein the object is a local coil.

12. At least one non-transitory computer-readable medium comprising instructions stored thereon, that when executed by one or more processors, cause the one or more processors to perform the method of claim 1.

13. A system for determining an orientation of a movable object relative to a B0 field magnet of a magnetic resonance tomography device in an xz-coordinate plane by a three-dimensional magnetic field strength sensor arranged at a fixed relative position on the object, the system comprising:
a first interface configured to receive B0 reference data of the B0 field magnet with characteristic magnetic field strengths for a number of xyz-coordinates;
a second interface configured to receive first measurement values of the magnetic field strength sensor at a first position, the first measurement values including at least field strength components of the B0 field;
a third interface configured to receive second measurement values of the magnetic field strength sensor at a second position, the second measurement values including at least field strength components of the B0 field; and
a controller in communication with the first, the second, and the third interfaces and configured to:
position the object at a first position outside the B0 field magnet with regard to a z-coordinate axis;
determine a first angle value based on the first measurement values of the magnetic field strength sensor at the first position and the B0 reference data;
position the object at a second position within the B0 field magnet with regard to the z-coordinate axis;
determine a second angle value based on the second measurement values of the magnetic field strength sensor at the second position and the B0 reference data;
correct the first angle value based on the second angle value to determine a corrected first angle value; and
provide the corrected first angle value in electronic form as a data file.

14. The system as claimed in claim 13, further comprising a three-dimensional acceleration sensor arranged with a fixed relative connection on the object and configured to provide an orientation of the object in relation to a y-coordinate axis at the first position and the second position, wherein the first and the second measurement values of the magnetic field strength sensor on the xz-coordinate plane are leveled based on the orientation of the object at the first position and the second position.

15. The system as claimed in claim 13, further comprising at least one memory storing B0 reference data of the B0 field magnet with characteristic magnetic field strengths for a number of xyz-coordinates and/or a number of angle values at the first position and corresponding corrected angle values at the first position.

16. An apparatus comprising:
one or more processors; and
memory storing instructions that, when executed by the one or more processors, cause the apparatus to:
position a movable object at a first position, with respect to a z-coordinate axis, outside a B0 field magnet of a magnetic resonance tomography device;
obtain first measurement values of a three-dimensional magnetic field strength sensor at the first position, the first measurement values including at least field strength components of a B0 field;
determine a first angle value based on the first measurement values of the magnetic field strength sensor at the first position and B0 reference data of the B0 field magnet with characteristic magnetic field strengths for a number of xyz-coordinates;
position the object at a second position within the B0 field magnet with respect to the z-coordinate axis;
obtain second measurement values of the magnetic field strength sensor at the second position, the second measurement values including at least field strength components of the B0 field;
determine a second angle value based on the second measurement values of the magnetic field strength sensor at the second position and the B0 reference data;
correct the first angle value based on the second angle value to determine a corrected first angle value; and
determine an orientation of the movable object relative to the B0 field magnet based on the corrected first angle value.
